(19) United States
(12) Patent Application Publication
(10) Pub. No.: US 2009/0082838 A1
(43) Pub. Date: Mar. 26, 2009

(54) LEFT-VENTRICULAR LEAD FIXATION DEVICE IN CORONARY VEINS

(75) Inventors: Bruce A. Tockman, Scandia, MN (US); Krishnan Ramaswamy, Roseville, MN (US)

Correspondence Address: FAEGRE & BENSON, LLP;ATTN: PATENT DOCKETING (32469), 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 11/862,164

(22) Filed: Sep. 26, 2007

(51) Int. Cl. A61N 1/372; A61B 18/00

(52) U.S. Cl. 607/126; 606/34; 607/122

(57) ABSTRACT

Methods for medical lead fixation in coronary veins according to embodiments of the present invention include advancing a lead body into a branch vessel of a coronary vein, inserting a fixation line and expandable anchor structure through the lead body, past a distal end of the lead body, and into the branch vessel, engaging a wall of the branch vessel with the expandable anchor structure, and coupling the fixation line with the lead body. Apparatus for medical lead fixation in a coronary vein according to embodiments of the present invention include a lead body having one or more electrodes, a fixation line, an expandable anchor structure coupled to the fixation line, the fixation line and anchor structure deployable through the lead body into the branch vessel, and a means for coupling the lead body to the fixation line.

### TECHNICAL FIELD

Embodiments of the present invention relate generally to medical devices and methods for placing a medical lead in a coronary vein. More specifically, embodiments of the present invention relate to devices and methods for anchoring a medical lead to an expandable anchor device in a branch vessel of a coronary vein.

### BACKGROUND

Cardiac function management systems are used to treat arrhythmias and other abnormal heart conditions. Such systems generally include cardiac leads, which are implanted in or about the heart, for delivering an electrical pulse to the cardiac muscle, for sensing electrical signals produced in the cardiac muscle, or for both delivering and sensing. The lead typically consists of a flexible conductor, defining a central channel or lumen, surrounded by an insulating tube or sheath extending from an electrode at the distal end to a connector pin (e.g., terminal pin) at the proximal end.

Cardiac lead placement may be accomplished by introducing the lead through a major blood vessel and advancing a distal end of the lead to a final destination in or near the heart. In the case of right atrial or right ventricular pacing the final destination is in the specific cardiac chamber. For left ventricular pacing the lead is often advanced from the right atrium, into the coronary sinus to reach a final destination within a branch vein residing on the epicardial surface of the left ventricle. To facilitate cannulation of the vasculature, it is often helpful to first advance a guiding catheter through the desired vascular path into the coronary sinus. One difficulty with implanting leads in this fashion is that the cardiac lead has a tendency to become dislodged from its desired location during or after lead implantation. For example, when a clinician withdraws the guiding catheter, the lead may dislodge or otherwise reposition. After the lead has been implanted, and until tissue in-growth ultimately fixes the lead at the desired site, the lead may have a tendency to migrate away from its original position over time, thus interfering with its reliability and performance.

### SUMMARY

According to embodiments of the present invention, coiling or shaped mechanisms are delivered through the lumen of a lead and advanced into a small distal vein where the intrinsic coil or pre-formed shape locks in the vessel and serves as an anchor. A tether or proximal extension between the lead and the anchor stabilizes the lead in the vessel, according to embodiments of the present invention.

According to embodiments of the present invention, a method for medical lead fixation in coronary veins includes advancing a lead body into a branch vessel of a coronary vein, inserting a fixation line through the lead body, past a distal end of the lead body, and into the branch vessel. According to such embodiments, the fixation line includes an expandable anchor structure, and the method may further include engaging a wall of the branch vessel with the expandable anchor structure and coupling the fixation line with the lead body. In some cases, the expandable anchor structure may include a core wire and an expandable coil pre-wound around the core wire, and engaging the wall of the branch vessel includes removing the core wire from the expandable coil to allow the expandable coil to expand and engage with the branch vessel. In other cases, the expandable anchor structure may include an expandable coil pre-wound around the fixation line, and engaging the wall of the branch vessel includes at least partially removing the fixation line from the expandable coil to allow the expandable coil to engage the branch vessel. In yet other cases, the expandable anchor structure may include a tube and an expandable coil within the tube, and engaging the wall of the branch vessel includes pushing the expandable coil out of the tube to engage the expandable coil with the branch vessel. The expandable coil may be include a pre-formed shape in its expanded shape, such as, for example, a helix, a corkscrew, a spiral, a tine, a sinusoid, and/or a hook.

Coupling the fixation line with the lead body may include capping a terminal pin of the lead body to restrict movement of the lead body in a proximal direction with respect to the fixation line. In one example, capping the terminal pin includes forming a head portion on the fixation line proximal of the terminal pin, the head portion having an outer dimension larger than the inner diameter of the lead body. In another example, capping the terminal pin includes folding the fixation line over the terminal pin and placing a lid over the fixation line and the terminal pin. In yet another example, capping the terminal pin includes folding the fixation line over the terminal pin and securing a band over the fixation line around the outside of the terminal pin. In a further example, capping the terminal pin includes flaring a proximal end of the fixation line to impart a diameter larger than the inner diameter of the lead body.

According to embodiments of the present invention, an apparatus for medical lead fixation in a coronary vein includes a lead body with one or more electrodes, a fixation line, an expandable anchor structure coupled to the fixation line at a distal end of the fixation line, the fixation line and the expandable anchor structure deployable through the lead body into a branch vessel of a coronary vein, and a means for preventing proximal migration of the lead body with respect to the fixation line. The expandable anchor structure may include a shape memory coil. In some cases, the shape memory coil may be pre-wound over an inner wire, such that retraction of the inner wire from the shape memory coil expands the shape memory coil against the coronary vein. In other examples, the shape memory coil may be contained by an outer tube, and an inner tube may be configured to push the shape memory coil out of the outer tube to expand the shape memory coil against the coronary vein.

Apparatus for medical lead fixation in a coronary vein may include a lead body having one or more electrodes, a fixation line, an expandable anchor structure coupled to the fixation line, and a means for deploying the lead body, according to embodiments of the present invention. The fixation line and the expandable anchor structure may be deployable through the lead body into a branch vessel of a coronary vein, and the expandable anchor structure may have an expanded configuration configured for engaging a wall of the coronary vein, according to such embodiments. Such embodiments according to the present invention may also include a means for coupling the fixation line to the lead body.

The lead body may include a first lumen and a second lumen, such that the first lumen is configured to receive the fixation line and the expandable anchor structure, and the second lumen is configured to receive the means for deploying the lead body. In some examples, the second lumen may be formed by an inner wall of the lead body, and the first lumen may extend within the second lumen. In other examples, the lead body includes a third lumen formed by an inner wall of the lead body.

According to some embodiments of the present invention, the fixation line diverges from the lead body distally of a terminal pin of the lead body. In some cases, the fixation line diverges from the lead body through a hole, and a knot may be tied in the fixation line outside of the hole to prevent proximal movement of the lead body with respect to the fixation line beyond the knot.

The expandable anchor structure may include a shape memory coil. According to some embodiments, the shape memory coil may be pre-wound over an inner wire, such that retraction of the inner wire from the shape memory coil expands the shape memory coil against the coronary vein. According to other embodiments, the shape memory coil may be contained within an outer tube during deployment of the anchor structure through the lead body, and an inner tube may be configured to push the shape memory coil out of the outer tube to expand the shape memory coil against the coronary vein. The expandable anchor structure and/or the shape memory coil may be made of a resorbable polymer, according to embodiments of the present invention.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a lead deployed in a patient's heart, according to embodiments of the present invention.

FIG. 2 illustrates a lead placed into a branch vessel of a coronary vein, according to embodiments of the present invention.

FIG. 3 illustrates an expandable anchor structure advanced through the lead in a branch vessel, according to embodiments of the present invention.

FIG. 4 illustrates an expanded anchor structure in a branch vessel, according to embodiments of the present invention.

FIG. 5 illustrates an anchor structure with a coil spring inside of a hypo tube, according to embodiments of the present invention.

FIG. 6 illustrates an anchor structure with a coil spring pre-wound over a core wire, according to embodiments of the present invention.

FIG. 7 illustrates a method for medical lead fixation in coronary veins, according to embodiments of the present invention.

FIG. 8 illustrates a pulse generator and terminal end of a lead, according to embodiments of the present invention.

FIG. 9 illustrates an enlarged side cross-sectional view of a terminal end tethering device, according to embodiments of the present invention.

FIG. 10 illustrates an enlarged side cross-sectional view of an alternative terminal end tethering device, according to embodiments of the present invention.

FIG. 11 illustrates an enlarged side cross-sectional view of an alternative terminal end tethering device, according to embodiments of the present invention.

FIG. 12 illustrates an enlarged side cross-sectional view of an alternative terminal end tethering device, according to embodiments of the present invention.

FIG. 13 illustrates an enlarged side cross-sectional view of an alternative terminal end tethering device, according to embodiments of the present invention.

FIG. 14 illustrates a partial side cross-sectional view of a multiple lumen configuration, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

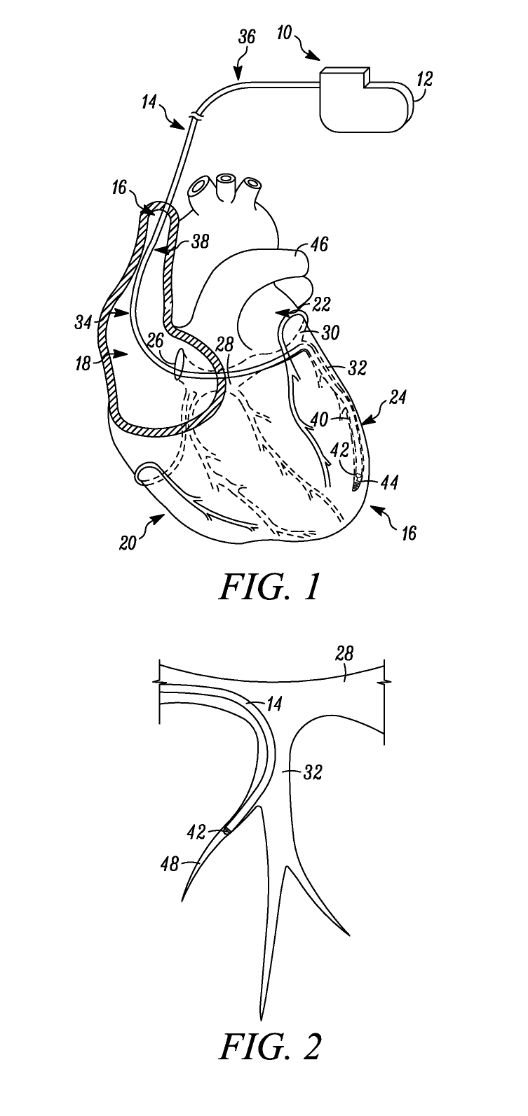

FIG. 1 is a schematic drawing of a cardiac rhythm management system 10 including a pulse generator 12 coupled to a lead 14 deployed in a patient's heart 16 from a superior vena cava 17. As is known in the art, the pulse generator 12 is typically implanted subcutaneously at an implantation location in the patient's chest or abdomen. As shown, the heart 16 includes a right atrium 18 and a right ventricle 20, a left atrium 22 and a left ventricle 24, a coronary sinus ostium 26 in the right atrium 18, a coronary sinus 28, and various cardiac branch vessels including a great cardiac vein 30 and an exemplary branch vessel 32.

As shown in FIG. 1, the lead 14 may include an elongate body 34 including a proximal region 36 and a distal region 38. The distal region 38 has a distal end 40 including an electrode 42 and terminating in an expandable anchor structure 44, according to embodiments of the present invention. To facilitate left ventricular pacing epicardially via a transvenous approach, leads 14 may be deployed in coronary veins 32 through the coronary sinus 28. In some cases, instability of leads 14 may result in extended procedure times, re-operation, loss of capture, phrenic nerve stimulation, and loss of resynchronization therapy. Embodiments of the present invention provide an expandable anchor structure 44 for secure fixation to a wall of the vessel 32.

Although FIG. 1 depicts the lead 14 as part of a cardiac rhythm management system 10 with an electrode 42, the lead 14 may alternatively include one or more sensors and/or one or more electrodes 42, and may couple the one or more sensors with a monitor instead of and/or in addition to the pulse generator 12.

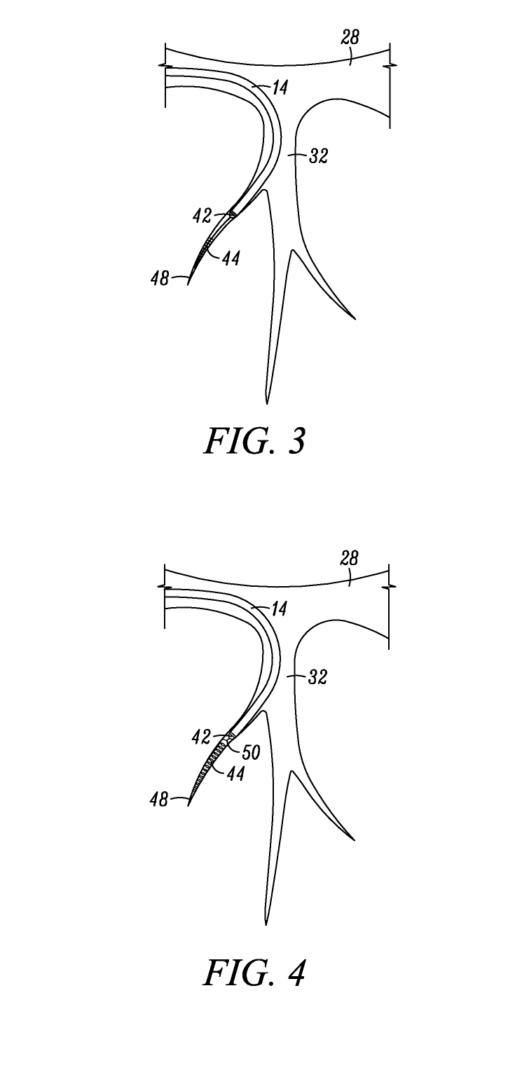

FIG. 2 illustrates a placement of the lead 14 through the coronary sinus 28 and into a branch vessel 32 of a cardiac vein, according to embodiments of the present invention. The lead 14 may be wedged into the branch vessel 32. According to some embodiments of the present invention, the lead 14 may be wedged within a further branch vessel (i.e., tributary) 48 to further enhance placement and/or anchoring of the lead 14. Next, as depicted in FIG. 3, after the lead is placed at its target location, an anchoring structure 44 in its unexpanded state may be advanced through the lead 14, out through the end of the lead 14 (near electrode 42, for example), and out into the branch vessel 48, according to embodiments of the present invention. As the anchoring structure 44 in its unexpanded state is narrow enough to be deployed through a lumen within the lead 14, the anchoring structure 44 in its unexpanded state is also narrow enough to be advanced further into the branch vessel 48 than the lead 14 itself.

FIG. 4 illustrates the expandable anchoring structure 44 in its expanded state, according to embodiments of the present invention. In the embodiment depicted in FIG. 4, coils of the anchoring structure 44 have expanded to engage with and exert a radial force on the sidewalls of the branch vessel 48, thereby anchoring the anchoring structure 44 within branch vessel 48. Although not shown in FIG. 4, the anchoring structure 44 may be inserted into very small reaches of the distal vein 48 not much larger than a guide wire (e.g. approximately fourteen thousandths of an inch), according to embodiments of the present invention. A fixation line 50 is coupled with the anchor structure 44, and may extend through a lumen of the lead 14 from the anchor structure 44 on one end to the proximal end 36 of the lead 14 on the other end. Alternatively, the fixation line 50 may be attached to the lead 14 at a location other than the proximal end 36, according to embodiments of the present invention. As used herein, the term “coupled” is used in its broadest sense to refer to elements which are connected, attached, and/or engaged, either directly or integrally or indirectly via other elements, and either permanently, temporarily, or removably.

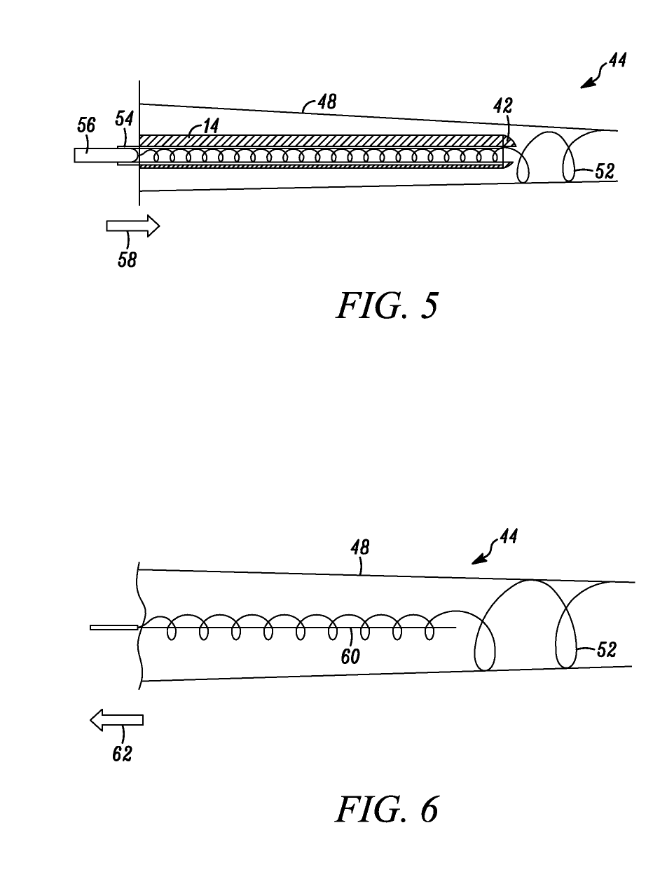

FIGS. 5 and 6 illustrate deployment mechanisms for anchor structures 44, according to embodiments of the present invention. FIG. 5 illustrates a spring coil 52 inside a tube 54. The tube may be a polymer and/or metal hypo tube 54, similar in size and flexibility to a guide wire, according to embodiments of the present invention. Once the hypo tube 54 is delivered ahead of the lead 14 to a desired location of the branch vessel 48, the tube 54 may be pulled back to deploy the spring coil 52 at the distal end of the lead 14 (near electrode 42). Alternatively, an inner tube 56 within outer tube 54 may be used to push the spring coil 52 in the direction of arrow 58 and out of the outer tube 54, thereby causing the spring coil 52 to expand to engage the side walls of the branch vessel 48, according to embodiments of the present invention. The proximal end of spring coil 52 may be coupled or otherwise secured to the terminal pin of the lead 14 near the proximal end 36, according to various embodiments of the present invention. The tube 54 may be extended from the lead body 14 prior to pushing the spring coil 52 out of the tube 54, in some embodiments of the present invention. In other embodiments, the inner lumen of the lead 14 itself serves to retain the spring coil 52 in its unexpanded state, and tube 56 may serve to push spring coil 52 in the direction 58 out of the lead 14.

FIG. 6 illustrates an alternative deployment mechanism for the anchor structure 44, according to embodiments of the present invention. The anchor structure 44 depicted in FIG. 6 includes a pre-wound coil 52 over a core wire 60. Once the anchor structure 44 is advanced ahead of the lead 14 to the desired location in the branch vessel 48, the core wire 60 is pulled back or removed in the direction indicated by arrow 62, allowing the coil 52 to reform to its intrinsic shape and engage the vessel 48 walls, according to embodiments of the present invention. According to various embodiments, the spring coil 52 of FIGS. 5 and 6 is a metal coil constructed from stainless steel and/or a shape memory alloy (e.g., nitinol). According to other embodiments of the present invention, the spring coil 52 and/or other elements of the anchor structure 44 may be made with a polymer, such as, for example, a resorbable polymer.

According to embodiments of the present invention, the proximal end of the spring coil 52 may be constructed of a metal or polymer hypo tube that can be secured at the terminal pin near pulse generator 12 by various methods, described below. According to some embodiments, some part such a metal or polymer hypo tube remains in the lead 14 and provides a connection to the anchor structure 44. In some cases, the spring coil 52 is coupled with a fixation line 50 which extends within the lead 14 to the proximal end 36; in other cases, the spring coil 52 itself is formed integrally with the fixation line 50. The fixation line may be soldered or otherwise attached to the spring coil 52, for example. According to some embodiments of the present invention, the anchor structure 44 and/or the fixation line are constructed entirely out of polymers. The anchor structure 44, for example, may be constructed of a resorbable polymer to permit extraction of the lead 14 after insertion.

The fixation line 50 may widen as it extends from near the anchor structure 44 to near the proximal end 36; for example, the fixation line 50 may gradually increase in diameter as it approaches proximal end 36 or may be coupled with a hypo tube which may, in turn, be coupled with the terminal pin of the lead 14 as described below. According to some embodiments of the present invention, the fixation line 50 itself is the core wire 60. The core wire 60 and/or the fixation line 50 may taper near its distal end. According to some embodiments of the present invention, the spring coil 52 also has a pre-formed macro shape such as, for example, a corkscrew or spiral or helix. When the spring coil 52 is allowed to expand, it not only engages the side walls of branch vessel 48, but it also assumes a particular macro shape to further enhance anchoring. The spring coil 52 may also be configured to assume other macro shapes. For example, the spring coil 52 may be configured to assume two-dimensional shapes such as sinusoidal shapes, tines, and/or hooks, according to embodiments of the present invention.

According to some embodiments of the present invention, the fixation line 50 and anchor structure 44 may be a constructed of a continuous polymer hypo tube in which the distal end has a pre-formed shape to engage the inner walls of the branch vessel 48. Cuts or kerfs could be made in the pre-formed section so that, with a core wire 60, the hypo tube distal end exhibits the performance characteristics of a guide wire. Once the core wire 60 is removed from the pre-formed section, the pre-formed section expands to its original macro shape, similar to the spring coil 52 embodiments described, above. According to such embodiments, the pre-formed section may be constructed of a resorbable polymer.

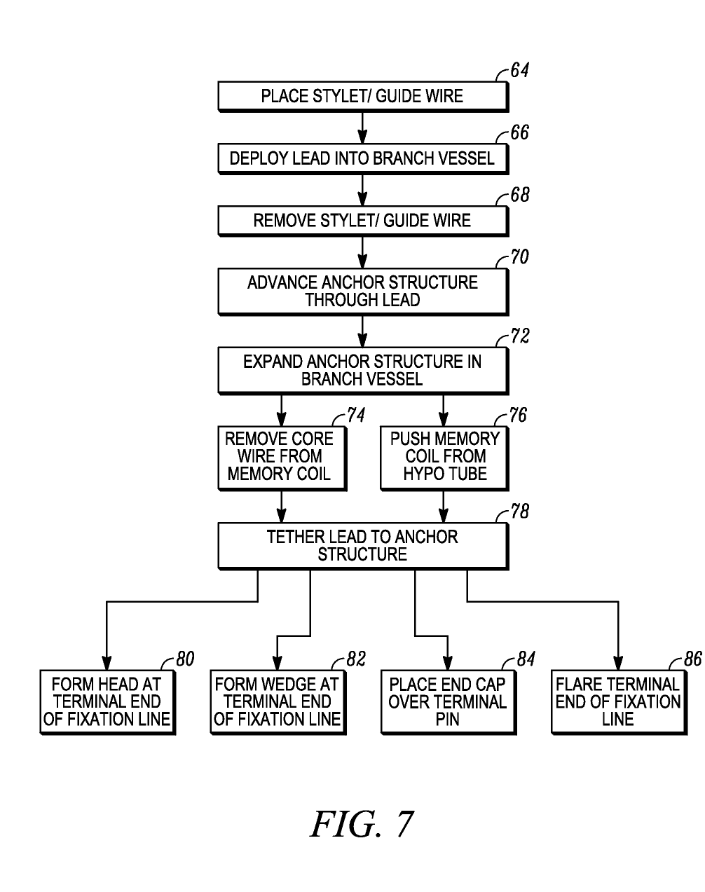

FIG. 7 illustrates a method for medical lead fixation in coronary veins, according to embodiments of the present invention. As is typically done in lead placement, a stylet may be loaded in the lead 14 to shape and/or deflect the lead 14 and push the lead 14 forward, or a guide wire may be advanced through the coronary sinus ostium 26, through the coronary sinus 28, into a branch vessel 32, and further into a tributary 48, according to embodiments of the present invention (block 64). Next, the lead 14 may be deployed into the branch vessel 32 (block 66); for example, the lead 14 may be deployed into the branch vessel 48 over a guide wire by an over-the-wire technique, according to embodiments of the present invention. The lead 14 may also be wedged into the branch vessel 48 to further enhance anchoring and/or to improve contact between the electrode 42 and the tributary 48, according to embodiments of the present invention.

Once the lead 14 is placed in its desired position, the stylet or guide wire used to place the lead 14 may be removed (block 68); for example, the guide wire may be removed by drawing it back through a lumen of the lead 14, according to embodiments of the present invention. The anchor structure 44 may then be advanced through the lead 14 (block 70). According to some embodiments of the present invention, a fixation line 50 is coupled with the anchor structure 44 and may be advanced through the lead 14 behind the anchor structure 44. The anchor structure 44 may be advanced through the distal end of the lead 14 and further into the branch vessel 48, at which point the anchor structure 44 may be expanded in the branch vessel 48, according to embodiments of the present invention (block 72). As described above, the expansion of the anchor structure 44 may be accomplished by removing the core wire 60 from the memory coil 52 (block 74) for deployment mechanisms similar to that of FIG. 6, or the spring coil 52 may be pushed from within the hypo tube 54 (block 76) for deployment mechanisms similar to that of FIG. 5, according to embodiments of the present invention. Based on the disclosure provided herein, one of ordinary skill in the art will recognize the various structures that may be deployed through the lead 14 and anchored in the branch vessel 48, including, but not limited to, a wire, a balloon, a stent, a hook, a tine, and/or a coil.

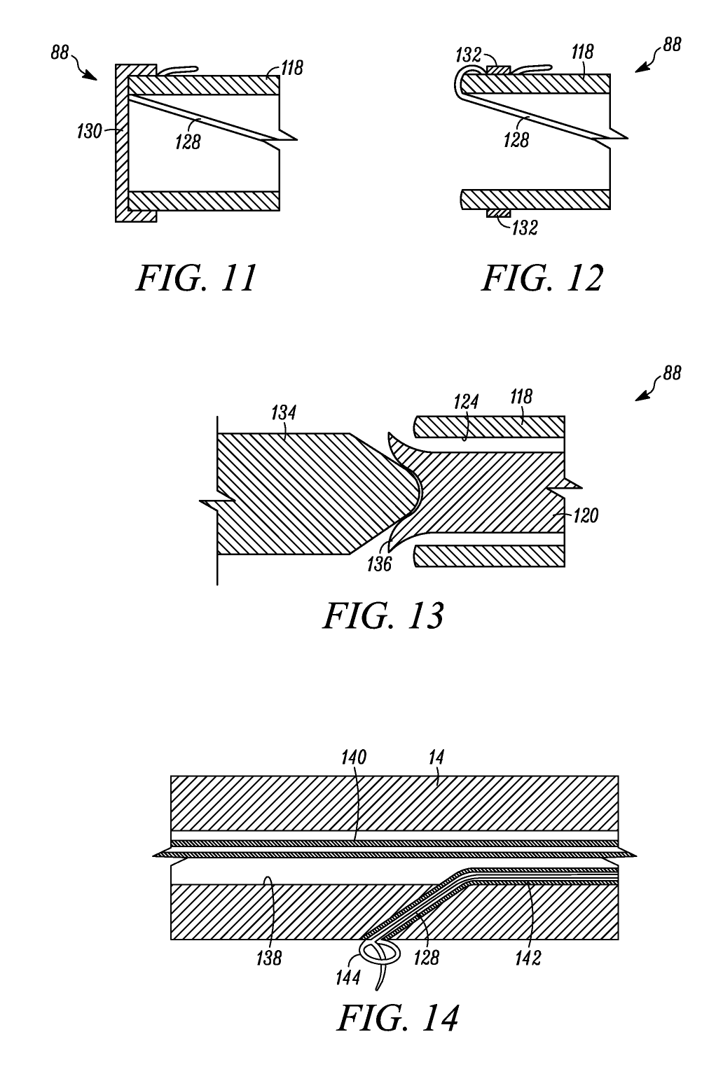

Once the anchor structure 44 has been expanded to secure it in the branch vessel 48, the lead 14 may be tethered to the anchor structure 44 (block 78). According to some embodiments of the present invention, the anchor structure 44 is tethered to the lead 14 by coupling the anchor structure 44 with a fixation line 50, and then coupling the fixation line 50 to a terminal pin of the lead 14 near the proximal end 36. According to some embodiments, the fixation line 50 is a hypo tube or similar device. The anchor structure 44 may be coupled with the lead 14 in various ways; for example, a head may be formed at the terminal end of the fixation line 50 (block 80), such that the head of the fixation line 50 is larger than an inner diameter of the lumen of the lead 14, as illustrated in FIG. 9. As another example, a wedge may be formed at the terminal end of the fixation line 50 (block 82), as illustrated in FIG. 10; as yet other examples, the fixation line 50 may be folded over the proximal end of the terminal cap, and an end cap placed over the terminal pin and the fixation line 50 (block 84), as illustrated in FIG. 11, or a band (e.g. a rubber band) may be placed over the terminal pin and the fixation line 50, as illustrated in FIG. 12. For embodiments in which the fixation line 50 is a hypo tube or a polymer strand, a tool may be used to flare the end of the fixation line 50 (block 86), as illustrated in FIG. 13. According to other embodiments, the anchor structure 44 is tethered or otherwise coupled to the inside of the lead 14 at or near the distal end of the lead 14.

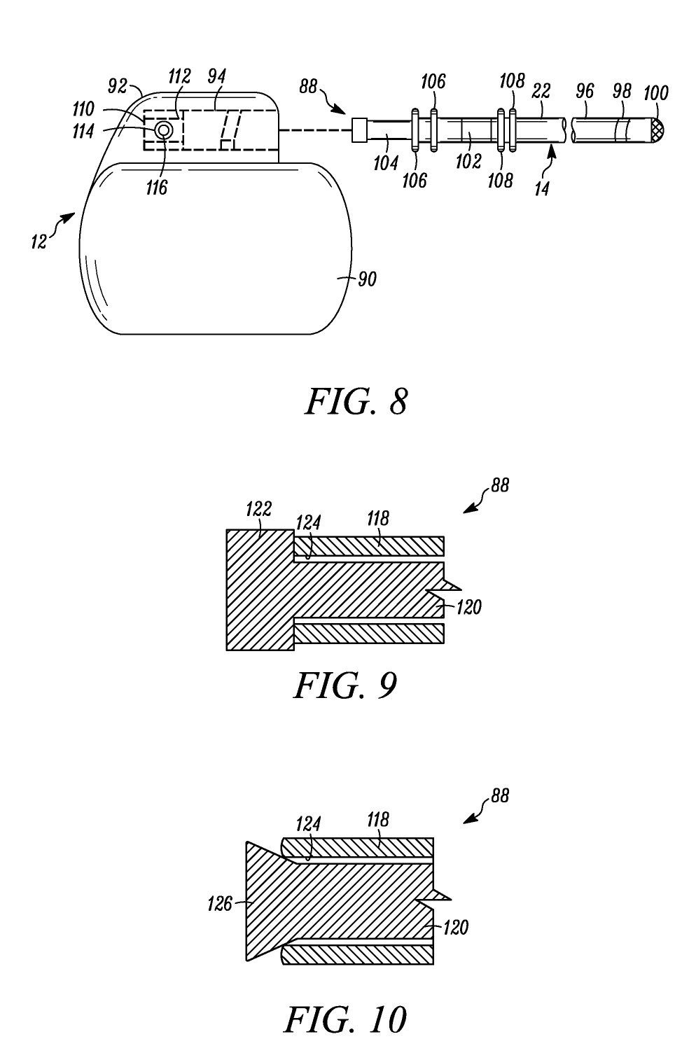

FIG. 8 illustrates a pulse generator 12 and a terminal end 88 of the lead 14, according to embodiments of the present invention. A hermetically sealed housing 90 may contain a battery and electronic circuitry for producing pulses of preprogrammed amplitude, duration and repetition rate dictated by a microprocessor-based controller forming a part of the electronic circuit contained within the hermetically sealed housing 90. The pulse generator 12 has a molded plastic connector 92 affixed to it and formed longitudinally in the connector is a lead receiving bore 94 into which the terminal portion 88 of a medical lead 14 is inserted, according to embodiments of the present invention.

The lead 14 includes an elongated, flexible, lead body 96 having one or more electrodes 98, 100 near or at its distal end. These electrodes are connected by elongated flexible conductors (not shown) that extend through the lead body 96 and are insulated from one another. The conductors connect to contacts 102, 104 disposed on the proximal terminal 88 of the lead. Sealing rings 106, 108 on the lead 14 interface with the wall of the bore 94 to prevent ingress of body fluids into the bore 94 of the connector 92.

The implantable device 12 may include a locking mechanism in the connector for preventing disengagement of the contact areas 102, 104 on the lead terminal 88 from mating contacts contained in the bore 94. A typical lead lock includes a block of metal 110 having a longitudinal bore 112 formed therethrough, that bore being intersected by a transversely extending threaded bore 114. Fitted into the threaded bore 114 is a setscrew 116. An elastomeric plug is fitted into the bore 114, again to prevent ingress of body fluids into the interior of the connector. At the time of implant, the setscrew 116 is tightened using a torquing tool inserted through the elastomeric plug so as to tightly press the contact 104 on the lead against the wall of the bore 112, according to embodiments of the present invention. The lead terminal 88 may also be referred to as a terminal pin, according to embodiments of the present invention.

FIG. 9 illustrates an enlarged view of the lead terminal 88 of FIG. 8, according to embodiments of the present invention. A terminal pin 118 surrounds the proximal end of the fixation line 120. Although the fixation line 120 is shown in FIG. 9 as a solid tube, fixation line 120 may also be, for example, a wire and/or a hollow hypo tube, according to embodiments of the present invention. Fixation line 120 has formed at its proximal end a head 122, an outer dimension of the head 122 being larger than the inner diameter 124 of the terminal pin 118, according to embodiments of the present invention. Such a configuration prevents the terminal pin 118, and thus the lead 14, from sliding proximally with respect to the fixation line, thus substantially hindering migration of the lead 14 and thus the electrodes 42 thereon. Similar to the head 122 of FIG. 9, the fixation line 120 of FIG. 10 has formed at its proximal end a wedge 126, an outer dimension of the wedge 126 being larger than the inner diameter of the terminal pin 118, according to embodiments of the present invention.

FIGS. 11 and 12 illustrate a fixation line 128 that more closely resembles a filament or a thread, according to embodiments of the present invention. FIG. 11 depicts the fixation line 128 folded over the terminal pin 118, after which an end cap 130 is inserted over the outside of the terminal pin 118 to hold the fixation line 128 in place, according to embodiments of the present invention. According to some embodiments of the present invention, excess fixation line 128 which protrudes from the end of the terminal pin 118 may be cut. According to some embodiments of the present invention, the end cap 130 forms a pressure fit over the end of the terminal pin 118; according to other embodiments, the end cap 130 threadably engages the terminal pin 118. FIG. 12 depicts the fixation line 128 folded over the terminal pin 118, after which a band 132, such as, for example, a rubber band, is secured around the terminal pin 118 to couple the fixation line 128 with the terminal pin 118, according to embodiments of the present invention.

According to alternative embodiments of the present invention, end cap 130 may include a hole (e.g., a “pinhole”) through which the fixation line 128 may be threaded and then tied in a knot to deter proximal movement of the lead 14 with respect to the fixation line 128. According to yet other alternative embodiments of the present invention, the terminal cap 118, the head 122, and/or the wedge 126 may include a groove or notch around which a thin filament fixation line 128 may be wound and/or tied prior to coupling the head 122 or the wedge 126 with the terminal pin 118.

FIG. 13 illustrates another terminal end cap according to embodiments of the present invention. Once the lead and the anchoring structure 44 have been placed as described above, a wedge tool 134 may be inserted into the proximal end of the fixation line 120, which may be a solid or hollow hypo tube according to embodiments of the present invention. The insertion of the wedge tool 134 creates flared edges 136, the flared edges 136 having an outer dimension larger than the inner diameter 124 of the terminal pin 118 to substantially prevent proximal movement or migration of the terminal pin 118 and thus the lead 14 with respect to the flared edges 136, according to embodiments of the present invention. The terminal end cap embodiments of FIGS. 9-13 effectively couple or tether the anchor structure 44 (via a fixation line 120, 128) to the lead 14 (via the terminal pin 118), while still allowing enough room for the terminal pin 118 to be inserted into the longitudinal bore 112 of the pulse generator 12, according to embodiments of the present invention.

According to some embodiments of the present invention, the lead 14 includes a single lumen to accommodate a stylet or guide wire during lead 14 placement. According to such embodiments, the stylet or guide wire is removed from the lead 14 prior to insertion of the anchor structure 44 and/or the fixation line 50 through the lumen of the lead 14. FIG. 14 illustrates an alternative lead 14 lumen configuration, according to embodiments of the present invention. The lead 14 normally has a single lumen 138 through which a stylet or guide wire may be deployed and then removed prior to insertion of the anchor structure 44. FIG. 14 shows that within the lumen 138, the lead 14 may include one or more other lumens 140, 142 to permit deployment of the anchor structure 44 even while the stylet or guide wire remains within the lead 14, according to embodiments of the present invention. For example, lumen 140 may be used to receive a stylet or a guide wire for maneuvering the lead 14 into place, while lumen 142 may be used to deploy the anchor structure 44 and/or fixation line 50, according to embodiments of the present invention.

As shown in FIG. 14, lumen 142 may optionally exit the main lumen 138 at a location distal of the terminal pin 118. According to these embodiments in which the lumen 142 for deployment of the anchor structure 44 diverges from the main lumen 138 before reaching the proximal end of the lead 14, the terminal end cap examples of FIGS. 9-13 may not apply because the fixation line 128 would not reach the terminal pin 118. However, the fixation line 128 may still be coupled to the lead 14 and/or secured in a similar way. For example, a knot 144 may be tied in the proximal end of the fixation line 128 to tether the lead 14 to the fixation line 128, according to embodiments of the present invention. According to other embodiments of the present invention, either one or the other of the lumens 140, 142 may be absent. For example, lumen 142 may be included to isolate the deployment of the anchor structure 44 from the stylet or guide wire, and the stylet or guide wire may simply be inserted through the main lumen 138 of the lead 14 next to the lumen 142, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method for medical lead fixation in coronary veins, the method comprising:
advancing a lead body into a branch vessel of a coronary vein;
inserting a fixation line through the lead body, past a distal end of the lead body, and into the branch vessel, wherein the fixation line comprises an expandable anchor structure;
engaging a wall of the branch vessel with the expandable anchor structure; and
coupling the fixation line with the lead body.

2. The method of claim 1, wherein the expandable anchor structure comprises a core wire and an expandable coil pre-wound around the core wire, and wherein engaging the wall of the branch vessel comprises removing the core wire from the expandable coil to allow the expandable coil to expand and engage with the branch vessel.

3. The method of claim 2, wherein the expandable coil comprises a pre-formed shape in an expanded state.

4. The method of claim 3, wherein the pre-formed shape is a shape selected from the group consisting of: a helix, a corkscrew, and a spiral.

5. The method of claim 1, wherein the expandable anchor structure comprises an expandable coil pre-wound around the fixation line, and wherein engaging the wall of the branch vessel comprises at least partially removing the fixation line from the expandable coil to allow the expandable coil to engage the branch vessel.

6. The method of claim 1, wherein the expandable anchor structure comprises a tube and an expandable coil within the tube, and wherein engaging the wall of the branch vessel comprises pushing the expandable coil out of the tube to engage the expandable coil with the branch vessel.

7. The method of claim 6, wherein the expandable coil comprises a pre-formed shape in an expanded state, the pre-formed shape selected from the group consisting of: a helix, a corkscrew, a spiral, a tine, a sinusoid, and a hook.

8. The method of claim 1, wherein coupling the fixation line with the lead body comprises capping a terminal pin of the lead body to restrict movement of the lead body in a proximal direction with respect to the fixation line.

9. The method of claim 8, wherein the lead body has an inner diameter at the terminal pin, and wherein capping the terminal pin comprises forming a head portion on the fixation line proximal of the terminal pin, the head portion having an outer dimension larger than the inner diameter.

10. The method of claim 8, wherein capping the terminal pin comprises folding the fixation line over the terminal pin and placing a lid over the fixation line and the terminal pin.

11. The method of claim 8, wherein capping the terminal pin comprises folding the fixation line over the terminal pin and securing a band over the fixation line around an outside of the terminal pin.

12. The method of claim 8, wherein the lead body has an inner diameter at the terminal pin, and wherein capping the terminal pin comprises flaring a proximal end of the fixation line to impart a diameter larger than the inner diameter.

13. An apparatus for medical lead fixation in a coronary vein, the apparatus comprising:
a lead body having one or more electrodes;
a fixation line;
an expandable anchor structure coupled to the fixation line at a distal end of the fixation line, the fixation line and the expandable anchor structure deployable through the lead body into a branch vessel of a coronary vein; and
a means for preventing proximal migration of the lead body with respect to the fixation line.

14. The apparatus of claim 13, wherein the expandable anchor structure comprises a shape memory coil, the apparatus further comprising:
an inner wire, the shape memory coil pre-wound over the inner wire, wherein retraction of the inner wire from the shape memory coil expands the shape memory coil against the coronary vein.

15. The apparatus of claim 13, wherein the expandable anchor structure comprises a shape memory coil, the apparatus further comprising:
an outer tube configured to contain the shape memory coil during deployment of the expandable anchor structure through the lead body; and
an inner tube configured to push the shape memory coil out of the outer tube, wherein pushing the shape memory coil out of the outer tube expands the shape memory coil against the coronary vein.

16. An apparatus for medical lead fixation in a coronary vein, the apparatus comprising:
a lead body having one or more electrodes;
a fixation line;
an expandable anchor structure coupled to the fixation line, the fixation line and the expandable anchor structure deployable through the lead body into a branch vessel of a coronary vein, wherein the expandable anchor structure has an expanded configuration configured for engaging a wall of the coronary vein; and
a means for deploying the lead body.

17. The apparatus of claim 16, further comprising:
a means for coupling the fixation line to the lead body.

18. The apparatus of claim 16, wherein the lead body comprises a first lumen and a second lumen, wherein the first lumen is configured to receive the fixation line and the expandable anchor structure, and wherein the second lumen is configured to receive the means for deploying the lead body.

19. The apparatus of claim 18, wherein the lead body comprises a third lumen, wherein the third lumen is formed by an inner wall of the lead body.

20. The apparatus of claim 18, wherein the fixation line diverges from the lead body distally of a terminal pin of the lead body.

21. The apparatus of claim 20, further comprising a hole through which the fixation line diverges from the lead body, and a knot tied in the fixation line outside of the hole, wherein an outer dimension of the knot is larger than an inner dimension of the hole.

22. The apparatus of claim 16, wherein the expandable anchor structure comprises a shape memory coil, the apparatus further comprising:
an inner wire, the shape memory coil pre-wound over the inner wire, wherein retraction of the inner wire from the shape memory coil expands the shape memory coil against the coronary vein.

23. The apparatus of claim 19, wherein the expandable anchor structure is made of a resorbable polymer.

24. The apparatus of claim 16, wherein the expandable anchor structure comprises a shape memory coil, the apparatus further comprising:
an outer tube configured to contain the shape memory coil during deployment of the expandable anchor structure through the lead body; and
an inner tube configured to push the shape memory coil out of the outer tube, wherein pushing the shape memory coil out of the outer tube expands the shape memory coil against the coronary vein.
